(19) United States
(12) Patent Application Publication
(10) Pub. No.: US 2008/0160586 A1
(43) Pub. Date: Jul. 3, 2008

(54) Process for the Purification of Tacrolimus

(75) Inventors: Walter Cabri, Rozzano (IT); Paolo Paissoni, Druento (IT); Jacopo Roletto, Torino (IT); Luca Morra, Avigliana (IT)

Correspondence Address: ROTHWELL, FIGG, ERNST & MANBECK, P.C., 1425 K STREET, N.W., SUITE 800, WASHINGTON, DC, 20005, US

(73) Assignee: Antibioticos S.p.A., Rodano (IT)

(21) Appl. No.: 11/718,415

(22) PCT Filed: Oct. 24, 2005
(86) PCT No.: PCT/EP05/11393

§ 371 Date: Aug. 7, 2007

(30) Foreign Application Priority Data
Nov. 3, 2004 (IT) MI2004A002098

(51) Int. Cl. C12P 17/16

(52) U.S. Cl. 435/118

(57) ABSTRACT

A process for the purification and recovery of Tacrolimus (I) (17-allyl-1,14-dihydroxy-12-[2-(4-hydroxy-3-methoxycyclohexyl)-1-methyvinyl]-23,25- dimethoxy-13,19,21,27-tetramethyl-11,28-dioxi-4-azatricyclo-[22.3.1.0^{4.9}]octacos-18-en-2,3,10,16-tetraone), starting from Streptomyces sp fermentation broth. The process is particularly advantageous in terms of productivity and selectivity of the separation of impurities.

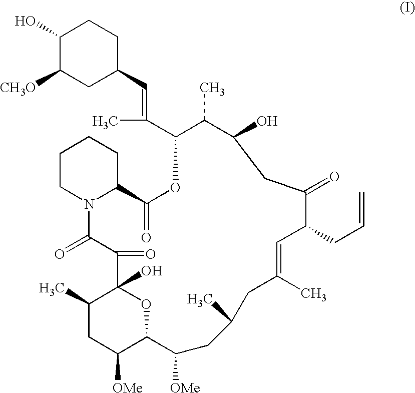

### FIELD OF THE INVENTION

The present invention relates in general to pharmacologically active immunosuppressant and antimicrobial tricyclic macrolides, in particular to a process for the recovery and purification of Tacrolimus (I)

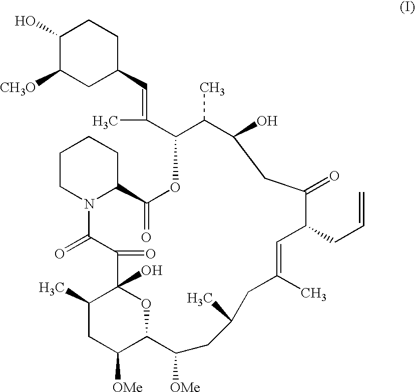

### BACKGROUND OF THE INVENTION

Tacrolimus (I) (17-allyl-1,14-dihydroxy-12-[2-(4-hydroxy-3-methoxycyclohexyl)-1-methylvinyl]-23,25-dimethoxy-13,19,21,27-tetramethyl-11,28-dioxy-4-azatricyclo-[22.3.1.0^{4.9}]octacos-18-en-2,3,10,16-tetraone) is a tricyclic macrolide produced by fermentation of Streptomyces sp., which is used in the treatment of transplant rejection crisis, autoimmune diseases, infectious diseases and the like.

EP 0184162 discloses a process for the preparation of Tacrolimus and derivatives thereof through fermentation and chemical synthesis. In particular, fermentation with Streptomyces sp. produces, further to Tacrolimus, also the 17-ethyl-derivative (II) (17-ethyl-1,14-dihydroxy-12-[2-(4-hydroxy-3-methoxycyclohexyl)-1-methylvinyl]-23,25-dimethoxy-13,19,21,27-tetramethyl-11,28-dioxi-4-azatricylo-[22.3.1.0^{4.9}]octacos-18-ene-2,3,10,16-tetraone), commonly known as FK520

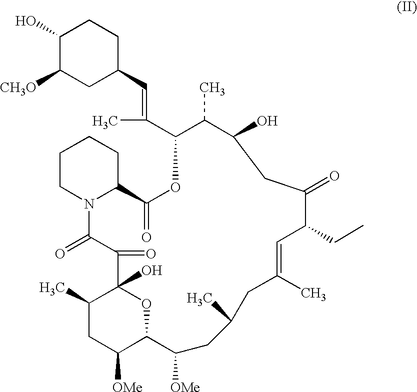

and the 17-propylderivative (III) (17-propyl-1,14-dihydroxy-12-[2-(4-hydroxy-3-methoxycyclohexyl)-1-methylvinyl]-23,25-dimethoxy-13,19,21,27-tetramethyl-11,28-dioxi-4-azatricyclo-[22.3.1.0^{4.9}]octacos-18-ene-2,3,10,16-tetraone)

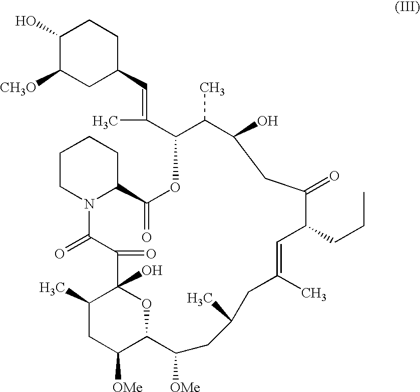

Besides the chemico-physical characterization of Tacrolimus and its by-products, EP 0184162 also discloses methods for its extraction, purification and recovery. In particular, the recovery of the products from fermentation broths is achieved by means of known extraction techniques, such as: use of conventional solvents to extract the activity from the broth or micelium; absorption/elution with ion-exchange anionic and cationic resins and non-ionic adsorbent resins; purification on conventional chromatographic supports such as silica gel, alumina and cellulose; decolourization with active charcoal, crystallization and recrystallization.

According to EP 0184162, extraction and recovery of Tacrolimus and by-products thereof from fermentation broths are carried out as follows:
extraction of the micelium and/or fermentation broth with a solvent (for example acetone and methanol);
purification through non-ionic adsorbent resins (in particular HP-20);
evaporation of the purified solution to an oil;
purification through silica gel (in particular silica gel grade 12 from Fuji Devison Co.), repeated two or three times to obtain a powder;
purification by preparative HPLC for the separation of the above-mentioned impurities.

The purification steps on non-ionic adsorbing resin and those on silica gel remove most of the compounds contained in the fermentation broth (i.e. substances produced by the microorganism, inorganic salts and substances deriving from the starting materials), whereas impurities (II) and (III) are removed by preparative HPLC, which has indeed poor productivity and applicability on an industrial scale.

U.S. Pat. No. 6,492,513 teaches to purify Tacrolimus from impurities (II) and (III) by ion-exchange cationic resins pretreated with silver salts (in particular silver nitrate). The use of silver salts for the separation of cis-trans isomers of unsaturated aliphatic acids with the same carbon atoms number is known in the literature (J. Chromatography, 149 (1978) 417). Silver salts form π-complexes with unsaturated compounds which are therefore separated according to their conformation. The process of U.S. Pat. No. 6,492,513 allows to separate Tacrolimus (which has a 17-allyl side chain) from the two impurities with 17-saturated side chains, since Tacrolimus is more retained than the other two impurities on cationic ion-exchange resins, due to the formation of the silver complex.

U.S. Pat. No. 6,576,135 teaches the separation of Tacrolimus from impurities (II) and (III) by means of non-ionic adsorbent resins, in particular with the following partial structure

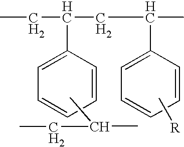

wherein R is a hydrogen or a halogen atom.

Several degradation products deriving from Tacrolimus are known in the literature (Y. Namiki et al. Cromatographia Vol. 40, No. 5/6 March 1995).

These degradation products are already present in the fermentation broth and can increase, depending on the working conditions, during the various extraction phases.

The processes disclosed in U.S. Pat. No. 6,492,513 and U.S. Pat. No. 6,576,135 allow the separation of Tacrolimus from impurities (II) and (III), but not from other degradation impurities.

### DETAILED DESCRIPTION OF THE INVENTION

It has now been found that Tacrolimus can be conveniently purified from degradation impurities as silver complex (IV)

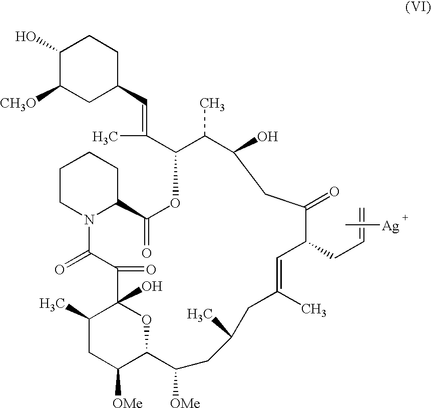

by means of C18 reverse phase silica.

In particular, the process of the invention comprises the dissolution of the fermentation product of Streptomyces sp in a water/organic solvent mixture containing silver ions and elution of the solution on a C18 reverse phase silica gel column.

Silver ions are released in the solution from silver salts, preferably silver nitrate or perchlorate. The concentration of silver ions preferably ranges from 0.05 to 1.30 mol/l, more preferably from 0.20 to 0.30 mol/l.

The organic solvent of the solvent mixture in which the product to purify is dissolved is an organic solvent wherein Tacrolimus is soluble, preferably selected from acetone, methanol and acetonitrile.

The amount of C18 reverse phase silica is 8 times the weight of crude product, preferably 12-14 times. Elution of the π-complex Tacrolimus-silver is carried out with the same solvent mixture used for the dissolution, gradually increasing the amount of organic solvent and collecting proper fractions from the chromatographic column. The concentration of silver ions in the eluent will range from 0.05 mol/l to 1.30 mol/l. The reverse phase silica is C18 silica with different granulometry, preferably 5-15 μm and 70-230 μm. The analytical method for the analysis of the eluted fractions is that disclosed in the literature (Y. Namiki et al. Cromatographia Vol. 40, No 5/6 March 1995) whereby it is possible to identify, by calculating the RRT, impurities (II), (III) and other degradation impurities.

The process of the invention can also comprise chromatographic purification on a non ionic resin and chromatographic purification on normal-phase silica gel, for example according to EP 0184162. These purification steps can be carried out either before or after the purification on C18 reverse phase silica gel. According to a particularly preferred embodiment, these further purifications can be carried out before, as hereinafter described in greater detail.

The fermentation broth or mycelium, suitably filtered, is extracted with organic solvents wherein Tacrolimus is soluble, for example ketones or alcohols, preferably acetone and methanol; the extraction product is subjected to adsorption chromatography on non ionic adsorbing resin, then to normal phase silica gel chromatography to purify Tacrolimus, impurities (II) and (III) and degradation products from other compounds deriving from the fermentation broth (substances produced by the microorganism, inorganic salts and substances deriving from starting materials). The resulting product is dissolved in an aqueous-organic solution and eluted on C18 reverse phase silica gel to recover the π-complex Tacrolimus-silver (IV), which is extracted with organic solvents in which Tacrolimus is soluble, for example ethyl acetate. The extraction product is concentrated and crystallized with known methods.

Purification on adsorbent resins is carried out using adsorbent resins available on the market, preferably those manufactured by Mitsubishi Chemical Corporation (series SP200 o SP800) or Rohm and Haas (series XAD). Preferred solvents are ketones or alcohols, more preferred are acetone and methanol.

Purification on normal phase silica gel is carried out using commercially available silica gels with different particle size, preferably 70-230 mesh. The solvents are preferably alkanes, esters, ketones and alcohols, more preferably n-hexane and ethyl acetate.

Extraction and crystallization are carried out according to the procedures for solvent extraction and recovery of Tacrolimus disclosed in the literature. Preferably, the solution containing the purified π-complex Tacrolimus-silver is concentrated under vacuum to remove the organic solvent and subsequently extracted with 0.5-3 volumes of organic solvent, preferably ethyl acetate. The organic phase is washed with 1 volume of deionized water for 2-3 times and subsequently concentrated to small volume. After dissolution of the resulting solution in an organic solvent, preferably acetonitrile, Tacrolimus precipitates as monohydrate crystals by addition of deionized water. The resulting crystals are characterized by high purity (HPLC area %>99% according to the HPLC method reported in Y. Namiki et al. Chromatographia Vol. 40, No 5/6 March 1995).

The process of the invention is particularly advantageous over known processes in terms of productivity, selectivity of the separation of the impurities and quality of the finished product. As regards productivity, the process of the invention requires an amount of chromatographic carrier (C18 reverse phase silica) per unit of crude product markedly lower (about 5-8 times) than that disclosed in U.S. Pat. No. 6,576,135 (wherein the chromatographic carrier is HP20ss). The percentage weight ratio of crude product to C18 reverse phase silica is 5-8%, while in the process of U.S. Pat. No. 6,576,135 the percentage ratio of crude product to chromatographic carrier HP20ss is 1%. The higher amount of product per weight unit of chromatographic carrier allows remarkable improvements in terms of productivity and costs on an industrial scale. The amount of finished product being the same the volumes in the purification phase are reduced by 5-8 times and as a consequence the costs due to silver salts (in particular AgNO₃) are also reduced.

Therefore, a single chromatographic step on C18 reverse phase silica provides a highly pure finished product on an industrial scale.

The invention will be now illustrated in greater detail by means of some examples.

### EXAMPLES

### Example 1

### Extraction and Purification on Adsorbing Resin

50 liters of fermentation broth are added with 50 liters of acetone and 1 kg of filtration adjuvant Dicalite. After stirring at room temperature for one hour the slurry is filtered. The resulting clear solution is absorbed on 2 liters of adsorbing resin XAD16 (manufactured by Rohm and Haas). The activity is eluted with 6 liters of 25/75 water/acetone. The resulting solution is concentrated to remove acetone. The aqueous phase (1.5 liters) is extracted with 1.5 liters of ethyl acetate. The phases are separated and the organic phase is concentrated to an oil.

### Example 2

### Purification on Silica Gel

The oily phase is added with 180 g of silica gel (0.063-0.200 mm Merck) and 180 ml of ethyl acetate. The mixture is stirred and subsequently evaporated to a powder, which is loaded onto a column containing 1 litre of silica gel (0.063-0.200 mm Merck) in n-hexane. Purification is accomplished eluting with 4 liters of n-hexane, then 4 litres of 75/25 n-hexane/ ethyl acetate and finally 10 litres of ethyl acetate. The eluted fractions are collected and each of them is analyzed by HPLC on a C18 column with water/acetonitrile as the eluant. Activity-enriched fractions are pooled and concentrated to obtain a white-yellowish solid (12 g).

### Example 3

### Dissolution and Purification of the π-Complex Tacrolimus-Silver

The solid of example 2 (12 g, containing 8.5 g of Tacrolimus), is dissolved in 400 ml of a 50/50 water/acetone solution containing 30 g of AgNO₃. The solution is passed through 200 ml of C18 reverse phase silica 15 μm (manufactured by Grace-Amicon). Afterwards, the column is eluted with 1000 ml of a 50/50 water/acetone solution containing 51 g of AgNO₃ and finally with 250 ml of a 20/80 water/acetone solution. The eluate is divided into fractions which are analyzed according to the analytical method reported in the Y. Namiki et al. Chromatographia Vol. 40, No 5/6 March 1995. The following table reports the variation of the Tacrolimus concentration and of the impurities during the various purification steps on C18 reverse phase silica.

Fractions 2, 3, 4 and 5 are combined and concentrated to 400 ml. 400 ml ethyl acetate is added, then the organic phase is separated and washed with 400 ml deionized water for 3 times. The organic phase is concentrated to small volume (10-15 ml).

### Example 4

### Recovery of Tacrolimus

The solution obtained according to example 3 is added with 700 ml acetonitrile. 1200 ml deionized water is slowly added (1-2 hours) at a temperature of 25° C. and the solution is cooled to 5° C., then allowed to stand at this temperature for 12-14 hours. After filtration 7.0 g Tacrolimus is obtained with high purity (HPLC Area %>99%).

TABLE

| | | | | Σ HPLC Area % |
|---|---|---|---|---|
| Purification phase, | HPLC Area % | HPLC Area % | HPLC Area % | Degradation |
| step 5) | Tacrolimus 1 | Impurity 2 | Impurity 3 | Impurity |
| Starting solution | 91.00% | 3.70% | 2.20% | 3.10% |
| Fraction 1: | 91.20% | 0.00% | 0.00% | 8.80% |
| H₂O/acetone 50/50 + AgNO₃ | | | | |
| Fraction 2 | 99.05% | 0.00% | 0.00% | 0.95% |
| H₂O/acetone 50/50 + AgNO₃ | | | | |
| Fraction 3 | 99.52% | 0.00% | 0.00% | 0.48% |
| H₂O/acetone 50/50 + AgNO₃ | | | | |
| Fraction 4 | 99.50% | 0.00% | 0.00% | 0.50% |
| H₂O/acetone 50/50 + AgNO₃ | | | | |
| Fraction 5 | 99.31% | 0.05% | 0.04% | 0.60% |
| H₂O/acetone 50/50 + AgNO₃ | | | | |
| Fraction 6 | 95.30% | 0.12% | 0.08% | 4.50% |
| H₂O/acetone 50/50 + AgNO₃ | | | | |
| Fraction 7 | 23.60% | 30.50% | 18.80% | 27.10% |
| H₂O/acetone 20/80 | | | | |

1. A process for the purification of Tacrolimus (I) 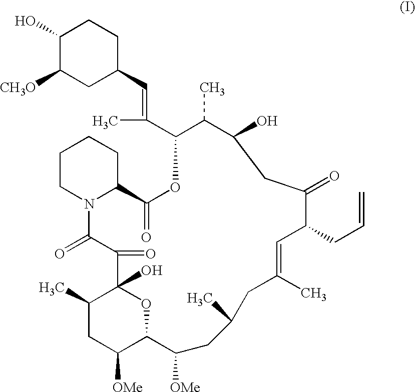
comprising the dissolution of the fermentation product of Streptomyces sp in a water/organic solvent mixture containing silver ions and the elution of the solution on a C18 reverse phase silica gel column.

2. The process as claimed in claim 1 wherein the silver ions are released from silver salts.

3. The process as claimed in claim 2 wherein the silver salt is silver nitrate or perchlorate.

4. The process according to claim 1 in which the silver ions concentration ranges from 0.05 to 1.30 mol/l.

5. The process as claimed in claim 4 wherein the concentration ranges from 0.20 to 0.30 mol/l.

6. The process according to claim 1 wherein the organic solvent is selected from acetone, methanol and acetonitrile.

7. The process according to claim 1, further comprising a chromatographic purification phase with a non-ionic resin and a chromatographic purification phase on normal phase silica gel.
